(19) United States
(12) Patent Application Publication
(10) Pub. No.: US 2015/0297097 A1
(43) Pub. Date: Oct. 22, 2015

(54) VASCULAR ACCESS EVALUATION AND TREATMENT

(71) Applicant: VOLCANO CORPORATION, San Diego, CA (US)

(72) Inventors: Bradley S. Matsubara, Escondido, CA (US); John Unser, Temecula, CA (US)

(21) Appl. No.: 14/596,710

(22) Filed: Jan. 14, 2015

Related U.S. Application Data
(60) Provisional application No. 61/927,032, filed on Jan. 14, 2014.

(51) Int. Cl. A61B 5/02; A61B 8/12; A61M 1/14; A61B 5/145; A61B 17/3207; A61M 39/02; A61B 5/026; A61B 5/021; A61B 5/00; A61M 25/10

(52) U.S. Cl. CPC A61B 5/02007; A61B 5/0084; A61B 8/12; A61B 5/489; A61B 5/742; A61M 1/14; A61M 25/104; A61B 17/3207; A61M 39/02; A61B 5/4839; A61B 5/026; A61B 5/021; A61B 5/14503

(57) ABSTRACT

The present invention generally relates to methods for assessing and treating dysfunctional vascular access sites, such as fistulas and grafts. According to certain aspects, methods of the invention include processing intraluminal image data of a vascular access site to characterize biological material present at the vascular access site, assessing the characterized biological material to identify a condition; and determining a therapeutic mode for treating the condition based on the assessment.

### CROSS-REFERENCE TO RELATED APPLICATIONS

This application claims the benefit of, and priority to, U.S. Provisional Application Ser. No. 61/927,032, filed Jan. 14, 2014, the content of which is incorporated by reference herein in its entirety.

### TECHNICAL FIELD

This application generally relates to characterizing and treating vascular access sites.

### BACKGROUND

Vascular access dysfunction is a leading cause of hospitalization and morbidity in the hemodialysis population. Currently, there are two main forms of permanent hemodialysis vascular access: native arteriovenous fistulas and synthetic grafts. Both forms of vascular access suffer from several complications. For example, surgically-created fistulas often fail to mature enough to support dialysis, and those that do mature often suffer from dilatation and stenosis. Vascular grafts do not suffer from initial maturation, but often form stenosis at the point of contact between the vessel and the graft. The stenosis, in both fistulas and grafts, is often formed from neointimal hyperplasia (e.g. smooth muscle cell migration and proliferation). Factors thought to contribute to the occurrence of neointimal hyperplasia include, for example, changes in blood flow hemodynamics along with damage to the vessel endothelium, compliance differences between the graft and the blood vessel, and changes in blood vessel stress. In addition to neointimal hyperplasia, thrombi (i.e., blood clots) and atheroma deposits (i.e., plaque) tend to form in and block fistulas and vascular grafts, thus further contributing to complications with vascular access.

Despite these complications, vascular access creation for hemodialysis is the most commonly performed type of vascular surgery. In order to maintain the stability and function of the vascular access, several interventional procedures have been developed to control stenosis and remove thrombotic and/or atheroma material at the vascular access site. The interventional procedures include, for example, performing an angioplasty, introducing a therapeutic agent to the vascular access site, ablating tissue at the vascular access site, performing an atherectomy, banding the vascular access site, introducing a vascular wrap, etc.

Digital subtraction angiography (DSA) is the current standard for diagnosing vascular access dysfunction and determining the appropriate interventional procedure for treatment. DSA involves injecting contrast material into an artery or vein, and then producing fluoroscopic images of the target area. The fluoroscopic images are then manipulated, via a computer, to subtract an image made with the contrast material from a post-injection image made without the contrast material. The subtraction eliminates features such as soft tissue, bones, etc. that are present in both the contrast and non-contrast images. The resulting image provides a prominent and clear view of the blood vessels containing the contrast material.

While DSA proves useful for determining the location of vessel occlusions and stenosis, the generated images do not provide insight, for example, as to the type or extent of the stenosis or thrombus causing vascular access dysfunction. As a result, one is not able to distinguish between conditions requiring surgical intervention and those that may be treated medicinally. This lack of insight provides limited guidance as to which type of surgical intervention would best treat the vascular access dysfunction.

### SUMMARY

Methods of the invention utilize intravascular imaging to guide diagnosis and course of treatment of dysfunctional vascular access sites (e.g., fistulas and grafts). The intravascular images provide direct visualization of the internal wall of the vascular access site as well as lesions within and surrounding the vascular access site. In some embodiments, spectral analysis tools are used in conjunction with imaging to further improve visualization of sclerosis, atheroma deposits, and/or thrombus morphology (i.e., virtual histology) contributing to vascular access dysfunction. This advantageously increases understanding of the health of the vascular access site and allows one to determine distinguishing between “at risk” lesions requiring surgical intervention and those that may be treated medicinally. Accordingly, the invention provides informed diagnostics and tailors course of treatment based on the informed diagnostics, thereby increasing the success of the interventional procedure subsequently used to treat vascular access dysfunction.

The invention includes methods of assessing intraluminal images of a vascular access site in order to identify a condition and determine course of treatment for the condition. Such methods include inserting an imaging catheter into a lumen of a vessel (i.e., vasculature) that has vascular access site identified as needing assessment and/or treatment. The vascular access site and surrounding vessel are then imaged, and tissue of the vessel are characterized, e.g., using spectral analysis such as virtual histology. The images and tissue characterization are then evaluated to identify a condition at the vascular image site and to determine a course of treatment for treating the condition. According to certain aspects, the method further provides for treating the condition in accordance with the pre-determined course of treatment.

By using spectral analysis and other processing techniques, any biological and/or non-biological (i.e. foreign) material present within or around the vascular access site can be characterized. Processing techniques for characterizing objects present in the image data may include, for example, determining the density of the biological material, determining the composition of the biological material, determining a blood-tissue border of the lumen of the vascular access site, and determining boundary of a graft forming the vascular access site.

A benefit of the present application is that the course of treatment can be tailored based on the intraluminal image assessment and tissue characterization. In certain aspects, methods of the invention provide for performing an interventional procedure in accordance with the intraluminal image assessment and tissue characterization. Suitable methods of treatment include one or more of performing an angioplasty procedure, introducing a therapeutic agent to the vascular access site, performing an atherectomy procedure, introducing a perivascular wrap, and a combination thereof. In certain embodiments, the course of treatment involves delivering an interventional catheter to the vascular access site, and treating a condition at the vascular access site with the interventional catheter. The condition may include sclerosis (e.g. neointimal hyperplasia), atheroma deposits (e.g. plaque), thrombosis (e.g. blood clots), or a combination thereof.

### BRIEF DESCRIPTION OF DRAWINGS

FIG. 1 illustrates a workflow for assessing and treating vascular access sites according to certain aspects.

FIG. 2 illustrates a phased-array ultrasound catheter according to certain embodiments.

FIG. 3 illustrates a rotational ultrasound catheter according to certain embodiments.

FIG. 4 depicts an image processing system for use with an imaging catheter.

FIG. 5 illustrates a pressure and flow guidewire for use with methods of the invention.

FIG. 6 illustrates a distal portion of an angioplasty catheter.

FIGS. 7-10 illustrate various cutting elements of atherectomy catheters.

### DETAILED DESCRIPTION

Methods of the invention can be used to assess, diagnose, and provide a course of treatment for any vascular access site, including fistulas and vascular grafts. An arteriovenous (AV) fistula is an induced native channel formed to connect an artery to a vein, whereas AV graft is an artificial connection that connects the artery to the vein. In addition, the term “fistula” is commonly used to generally describe both native and artificial connections between arteries and veins. As discussed in the background, both AV fistulas and grafts suffer from several complications (such as neointimal hyperplasia, atheroma deposits, thrombosis, and infection) that require monitoring and maintenance to ensure proper vascular access. Methods of the invention utilize intravascular imaging and tissue characterization to guide diagnosis and course of treatment of dysfunctional vascular access sites. The invention, according to certain aspects, provides for intraluminal imaging and tissue characterization of a vascular access site prior to an interventional procedure in order to optimize treatment decision making, thereby increasing the success of any interventional procedure used to treat a condition at the vascular access site.

While methods of the invention are described with regard to treating vascular access sites, methods of the invention can be utilized to assess and treat other fistulas. For example, fistulas formed in the respiratory system, digestive system, and circulatory system.

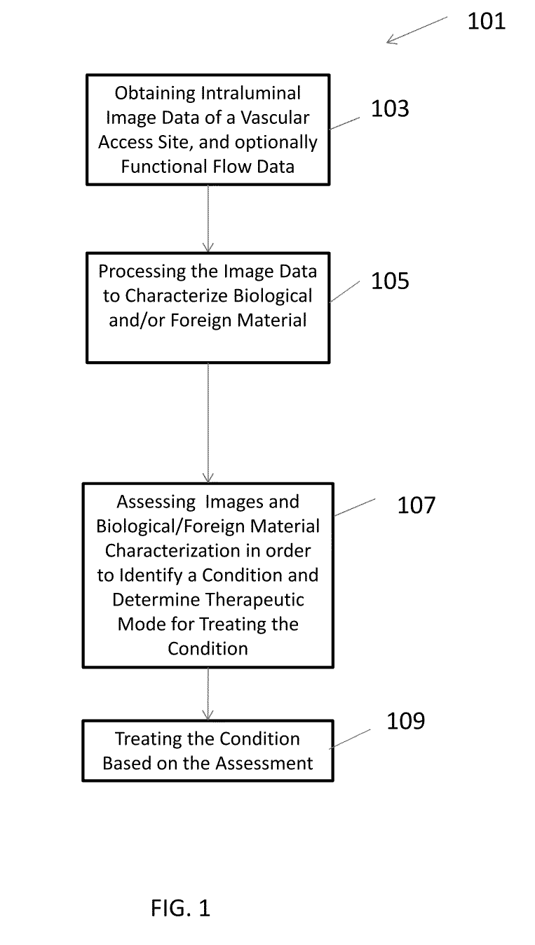

FIG. 1 illustrates an exemplary workflow 101 for evaluating and assessing vascular access sites for guiding diagnosis and course of treatment, according to certain embodiments. In the first step 103, intraluminal image data of a vascular access site are obtained. The intraluminal image data can be obtained with an intraluminal imaging catheter (or a guidewire). The imaging catheter can be used to image the vessels around and/or forming the vascular access site. For example, the imaging catheter can be used to image the vascular access site between a first blood vessel and a second blood vessel and to image portions of the first and second blood vessels near the vascular access site. In addition to obtaining intraluminal data, functional flow data may also be collected at step 103. Intraluminal devices, such as catheters and guidewires, for obtaining image and functional flow data are described in more detail hereinafter.

The image data is then processed to characterize biological material and/or foreign material (see step 105). For characterization of biological and/or foreign materials, a spectral analysis is applied to the image data, which involves examining the energy of the returned acoustic signal (or optical signal) at various frequencies. Tissue and object characterization beneficially allows one to determine the type and nature of a condition at the vascular access site. For example, in addition to identifying a stenosis, thrombosis, or infection at the vascular access site, the tissue characterization can assist in assessing risk of the condition, e.g., the severity of the neointimal hyperplasia, the presence and consistency of any atheroma material (e.g., level of calcification), and the severity of the thrombus.

Spectral analysis is useful for characterizing and determining the nature of the tissue and the presence of foreign objects. A plaque deposit, for example, will typically have different spectral signatures than nearby vascular tissue without such plaque, allowing discrimination between healthy and diseased tissue. Also a metal surface, such as a stent, will have a different spectral signal. Such signal processing may additionally include statistical processing (e.g., averaging, filtering, or the like) of the returned ultrasound signal in the time domain. The spectral analysis can also be used to determine the tissue lumen/blood border. Other signal processing techniques known in the art of tissue characterization may also be applied. Suitable types of signal processing for characterization, including spectral analysis, are described in more detail hereinafter.

The next step 107 in the method involves assessing the image data to identify a condition (e.g., stenosis, thrombus, infection) and determine a therapeutic mode for treating the condition. Because methods of the invention are able to determine the type and nature of the condition, one is able to tailor the subsequent therapeutic mode specific for that condition. This is in contrast to prior art techniques (e.g., Digital Subtraction Angiogram (DSA)) that only provide a generalized assessment of the condition without specifics.

Optionally, the method of the invention further includes treating the condition (step 109) based on the assessment step 107. With the information obtained from the assessment step 107, the treatment step 109 can be tailored to specifically treat the identified condition. In certain embodiments, the treatment step 109 includes introducing an interventional catheter to the vascular access site and performing one or more interventional procedures. Alternatively or in addition to, the treatment step may involve introducing one or more therapeutic agents to the vascular access site. Various treatments and interventional catheters are described hereinafter.

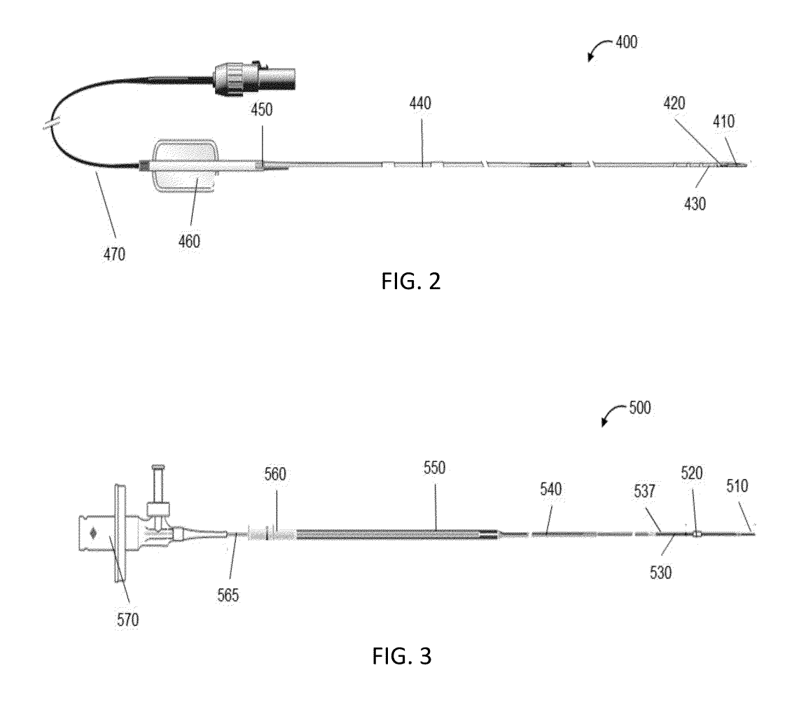

Exemplary imaging catheters that may be used to obtain image data for diagnosis and tissue/foreign object characterization are shown in FIGS. 2 and 3. The catheter shown in FIG. 2 is a generalized depiction of a phased array imaging catheter. Phased array imaging catheter 400 is typically around 200 cm in total length and can be used to image a variety of vasculature, such as coronary or carotid arteries and veins. Phased array catheter 400 can be shorter, e.g., between 100 and 200 cm, or longer, e.g., between 200 and 400 cm. When the phased array imaging catheter 400 is used, it is inserted into an artery along a guidewire (not shown) to the desired location (i.e. location of the vascular access site). Typically a portion of catheter, including a distal tip 410, comprises a guidewire lumen (not shown) that mates with the guidewire, allowing the catheter to be deployed by pushing it along the guidewire to its destination. The catheter, riding along the guidewire, can obtain images surrounding the vascular access site and within the vascular access site (e.g. within the fistula or AV graft).

An imaging assembly 420 proximal to the distal tip 410, includes a set of transducers that image the tissue with ultrasound energy (e.g., 20-50 MHz range) and a set of image collectors that collect the returned energy (echo) to create an intravascular image. The array is arranged in a cylindrical pattern, allowing the imaging assembly 420 to image 360° inside a vessel. In some embodiment, the transducers producing the energy and the collectors receiving the echoes are the same elements, e.g., piezoelectric elements. Because the phased array imaging catheter 400 does not have a rotating imaging assembly 420, the phased array imaging catheter 400 does not experience non-uniform rotation distortion.

Suitable phased array imaging catheters, which may be used to assess vascular access sites and characterize biological tissue located therein, include Volcano Corporation's Eagle Eye® Platinum Catheter, Eagle Eye® Platinum Short-Tip Catheter, and Eagle Eye® Gold Catheter.

FIG. 3 is a generalized depiction of a rotational imaging catheter 500 incorporating a proximal shaft and a distal shaft of the invention. Rotational imaging catheter 500 is typically around 150 cm in total length and can be used to image a variety of vasculature, such as coronary or carotid arteries and veins. When the rotational imaging catheter 500 is used, it is inserted into an artery along a guidewire (such as a pressure/flow guidewire) to the desired location. Typically a portion of catheter, including a distal tip 510, comprises a lumen (not shown) that mates with the guidewire, allowing the catheter to be deployed by pushing it along the guidewire to its destination.

An imaging assembly 520 proximal to the distal tip 510, includes transducers that image the tissue with ultrasound energy (e.g., 20-50 MHz range) and image collectors that collect the returned energy (echo) to create an intravascular image. The imaging assembly 520 is configured to rotate and travel longitudinally within distal shaft 530 allowing the imaging assembly 520 to obtain 360° images of vasculature over the distance of travel. The imaging assembly is rotated and manipulated longitudinally by a drive cable (not shown). In some embodiments of rotational imaging catheter 500, the distal shaft 530 can be over 15 cm long, and the imaging assembly 520 can rotate and travel most of this distance, providing thousands of images along the travel. Because of this extended length of travel, the speed of the acoustic waves through distal shaft 530 should ideally be properly matched, and that the interior surface of distal shaft 530 has a low coefficient of friction. In order to make locating the distal shaft 530 easier using angioscopy, distal shaft 530 optionally has radiopaque markers 537 spaced apart at 1 cm intervals.

Rotational imaging catheter 500 additionally includes proximal shaft 540 connecting the distal shaft 530 containing the imaging assembly 520 to the ex-corporal portions of the catheter. Proximal shaft 540 may be 100 cm long or longer. The proximal shaft 540 combines longitudinal stiffness with axial flexibility, thereby allowing a user to easily feed the catheter 500 along a guidewire and around tortuous curves and branching within the vasculature. The interior surface of the proximal shaft also has a low coefficient of friction, to reduce NURD, as discussed in greater detail above. The ex-corporal portion of the proximal shaft 540 may include shaft markers that indicate the maximum insertion lengths for the brachial or femoral arteries. The ex-corporal portion of catheter 500 also include a transition shaft 550 coupled to a coupling 560 that defines the external telescope section 565. The external telescope section 565 corresponds to the pullback travel, which is on the order of 150 mm. The end of the telescope section is defined by the connector 570 which allows the catheter 500 to be interfaced to an interface module which includes electrical connections to supply the power to the transducer and to receive images from the image collector. The connector 570 also includes mechanical connections to rotate the imaging assembly 520. When used clinically, pullback of the imaging assembly is also automated with a calibrated pullback device (not shown) which operates between coupling 2560 and connector 570.

The imaging assembly 520 produces ultrasound energy and receives echoes from which real time ultrasound images of a thin section of the blood vessel are produced. The transducers in the assembly may be constructed from piezoelectric components that produce sound energy at 20-50 MHz. An image collector may comprise separate piezoelectric elements that receive the ultrasound energy that is reflected from the vasculature. Alternative embodiments of the imaging assembly 520 may use the same piezoelectric components to produce and receive the ultrasonic energy, for example, by using pulsed ultrasound. Another alternative embodiment may incorporate ultrasound absorbing materials and ultrasound lenses to increase signal to noise.

Suitable rotational IVUS catheters, which may be used to assess vascular access sites and characterize biological tissue located therein, include Volcano Corporation's Revolution® 45 MHz Catheter.

Further, IVUS technology, for phased-array and rotational catheters, is described in more detail in, for example, Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, U.S. Pub. 2009/0284332; U.S. Pub. 2009/0195514 A1; U.S. Pub. 2007/0232933; and U.S. Pub. 2005/0249391 and other references well known in the art relating to intraluminal ultrasound devices and modalities.

In addition to IVUS, other intraluminal imaging technologies may be suitable for use in methods of the invention for assessing and characterizing vascular access sites in order to diagnose a condition and determine appropriate treatment. For example, an Optical Coherence Tomography catheter may be used to obtain intraluminal images in accordance with the invention.

OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable mirror, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference mirror, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

In advanced embodiments, the systems of the invention incorporate focused acoustic computed tomography (FACT), which is described in WO2014/109879, incorporated herein by reference in its entirety.

In yet another embodiment, the imaging catheter for use in methods of the invention is an optical-acoustic imaging apparatus. Optical-acoustic imaging apparatus include at least one imaging element to send and receive imaging signals. In one embodiment, the imaging element includes at least one acoustic-to-optical transducer. In certain embodiments, the acoustic-to-optical transducer is an Fiber Bragg Grating within an optical fiber. In addition, the imaging elements may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interferences in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

Exemplary optical-acoustic imaging assemblies are disclosed in more detail in U.S. Pat. Nos. 6,659,957 and 7,527,594, 7,245,789, 7447,388, 7,660,492, 8,059,923 and in U.S. Patent Publication Nos. 2008/0119739, 2010/0087732 and 2012/0108943.

In certain embodiments, angiogram image data is obtained simultaneously with the intraluminal image data obtained from the imaging catheters. In such embodiments, the imaging catheter may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

According to certain aspects of the invention, the obtained image data and/or functional flow data is processed to characterize biological material and/or foreign material at the vascular access site (as in step 105). The characterization allows one to determine with specificity any condition at the vascular access site and guides treatment of the treatment. The processing step may be performed by an image processing computer coupled to an imaging catheter. The imaging catheter may be directed coupled to the image processing computer or coupled to a system controller that allows for manipulation of the imaging catheter.

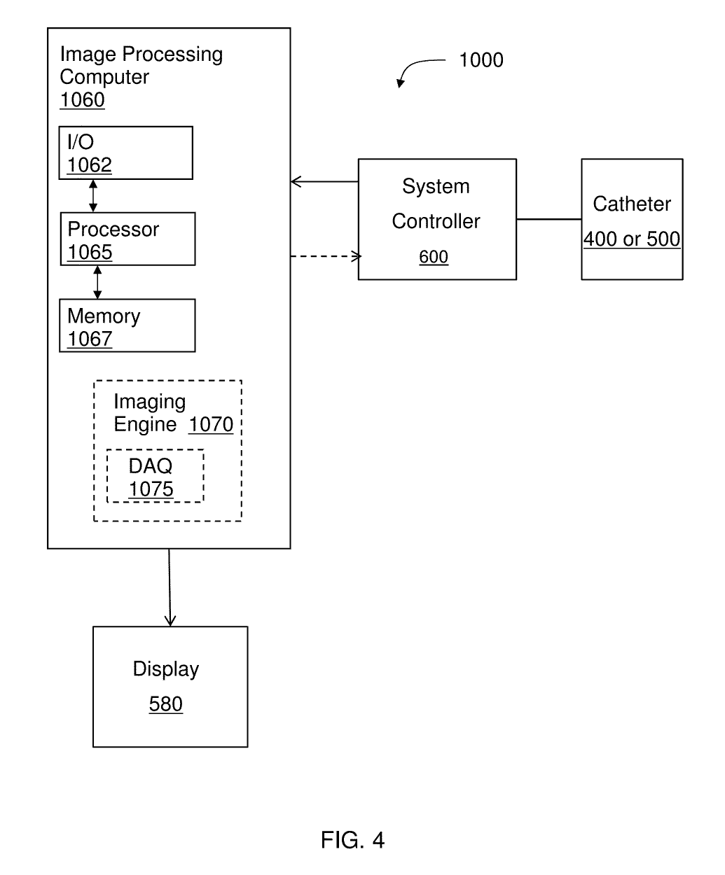

Referring now to FIG. 4, the imaging catheter 400, 500 may be coupled to and coordinated by a system controller 600. The system controller 600 may control the timing, duration, and amount of imaging. As shown in FIG. 4, the system controller 600 is additionally interfaced with image processing computer 1060. According to certain embodiments, the processor 1065 of the image processing computer 1060 performs tissue/blood characterization, thereby allowing the viewed and assessed images to be the basis for defining parameters for identifying a condition and developing a therapeutic mode for treating the condition. The systems 1000 also includes a display 580 and a user interface that allow a user, e.g. a surgeon, to interact with the images (including tissue characterization) and to control the parameters of the treatment.

As shown in FIG. 4, the system controller 600 is interfaced to an image processing computer 1060 that is capable of synthesizing the images and tissue measurements into easy-to-understand images. The image processing computer is also configured to analyze the spectrum of the collected data to determine tissue characteristics, a.k.a. virtual histology. As discussed in greater detail below, the image processing will deconvolve the reflected acoustic waves or interfered infrared waves to produce distance and/or tissue measurements, and those distance and tissue measurements can be used to produce an image, for example an IVUS image or an OCT image. Flow detection and tissue characterization algorithms, including motion-detection algorithms (such as CHROMAFLO (IVUS fluid flow display software; Volcano Corporation), Q-Flow, B-Flow, Delta-Phase, Doppler, Power Doppler, etc.), temporal algorithms, harmonic signal processing, can be used to differentiate blood speckle from other structural tissue, and therefore enhance images where ultrasound energy back scattered from blood causes image artifacts.

In certain embodiments, the image processing may additionally include spectral analysis, i.e., examining the energy of the returned acoustic signal at various frequencies. Spectral analysis is useful for determining the nature of the tissue and the presence of foreign objects. A plaque deposit or neointimal hyperplasia, for example, will typically have different spectral signatures than nearby vascular tissue without such plaque or neointimal hyperplasia, allowing discrimination between healthy and diseased tissue. Also a metal surface, such as a AV graft, will have a different spectral signal. Such signal processing may additionally include statistical processing (e.g., averaging, filtering, or the like) of the returned ultrasound signal in the time domain. The spectral analysis can also be used to determine the tissue lumen/blood border. Other signal processing techniques known in the art of tissue characterization may also be applied.

Other image processing may facilitate use of the images or identification of features of interest. For example, the border of a lumen may be highlighted or thrombus or plaque deposits may be displayed in a visually different manner (e.g., by assigning thrombus a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. In other embodiments, similar techniques are used to discern the extent and severity of the neointimal hyperplasia. Other measurements, such as flow rates or pressure may be displayed using color mapping or by displaying numerical values. In some embodiments, the open cross-sectional area of the lumen is colorized with red to represent the blood flux. Thus, by using virtual histology (spectral analysis), methods of the invention allow one to assess the type and severity of one or more conditions present within the vascular access site. In doing so, the need for treating the condition(s) and the type of treatment best suited for treating the condition may be determined.

In addition to the above disclosed systems, the following systems for detecting and characterizing plaque and biological tissue using virtual histology are disclosed in U.S. Pat. No. 6,200,268 entitled “VASCULAR PLAQUE CHARACTERIZATION” issued Mar. 13, 2001, U.S. Pat. No. 6,381,350 entitled “INTRAVASCULAR ULTRASONIC ANALYSIS USING ACTIVE CONTOUR METHOD AND SYSTEM” issued Apr. 30, 2002, U.S. Pat. No. 7,074,188 entitled “SYSTEM AND METHOD OF CHARACTERIZING VASCULAR TISSUE” issued Jul. 11, 2006, U.S. Pat. No. 7,175,597 entitled “NON-INVASIVE TISSUE CHARACTERIZATION SYSTEM AND METHOD” issued Feb. 13, 2007, U.S. Pat. No. 7,215,802 entitled “SYSTEM AND METHOD FOR VASCULAR BORDER DETECTION” issued May 8, 2007, U.S. Pat. No. 7,359,554 entitled “SYSTEM AND METHOD FOR IDENTIFYING A VASCULAR BORDER” issued Apr. 15, 2008, and U.S. Pat. No. 7,463,759 entitled “SYSTEM AND METHOD FOR VASCULAR BORDER DETECTION” issued Dec. 9, 2008.

In addition to tissue characterization, methods of the invention may also utilize functional flow measurements obtained at the vascular access site to assess the condition and determine course of treatment. Functional flow measurements allow one to determine pressure and flow differences at the vascular access site. Accordingly, imaging catheters of the invention may be equipped with one or more data collectors used to obtain functional flow measurements. Alternatively or in addition to, a guidewire with data collectors can be used alone or in combination with the imaging catheter to obtain the functional flow measurements (e.g., by using a pressure and/or flow guidewire and running the imaging catheter over that guidewire).

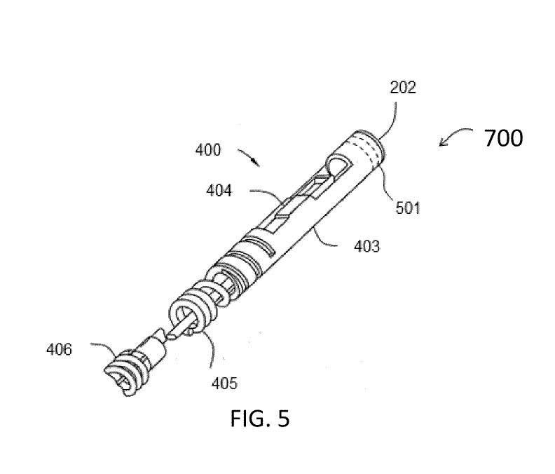

FIG. 5 shows a sensor tip 700 of a guidewire 401 that may be suitable to use with methods of the invention. Guidewire 401 will include one of pressure sensor 404 and ultrasound transducer 501. In general, guidewire 401 will sensor housing 403 for pressure sensor 404, ultrasound transducer 501, or both and may optionally include a radiopaque tip coil 405 distal to proximal coil 406. The radiopaque tip coil allows one to visualize the guidewire in angiograms.

Pressure sensor 404 can detect a lack of a pressure gradient, indicating that the fistula is not restrictive enough (i.e., if blood flows through the fistula too freely, it will not also flow to distal extremities of that limb of the body, leading to distal ischemia). It may be found, for example, that a ΔP of less than 20 or 30 mmHg is problematic. Pressure sensors and their use are described in U.S. Pub. 2009/0088650 to Corl. Ultrasound transducer 501 may include a forward-looking IVUS and can give the velocity of flow. Velocity data may be derived by the computer in the system from the Doppler frequency shifts detected in the ultrasound echo signals. Obtaining Doppler velocity is discussed in U.S. Pub. 2013/0303907 to Corl and U.S. Pub. 2007/0016034 to Donaldson. While the pressure sensor 404 and ultrasound transducer 501 are described as components of a guidewire, it is contemplated that the pressure sensor and ultrasound can transducer can also be incorporated into an imaging guidewire.

Guidewire 700 may comprise a flexible elongate element having proximal and distal ends and a diameter of 0.018″ or less as disclosed in U.S. Pat. No. 5,125,137, U.S. Pat. No. 5,163,445, U.S. Pat. No. 5,174,295, U.S. Pat. No. 5,178,159, U.S. Pat. No. 5,226,421, U.S. Pat. No. 5,240,437 and U.S. Pat. No. 6,106,476, all of which are incorporated by reference herein. Guidewire 700 can be formed of a suitable material such as stainless steel, Nitinol, polyimide, PEEK or other metallic or polymeric materials having an outside diameter for example of 0.018″ or less and having a suitable wall thickness, such as, e.g., 0.001″ to 0.002″. This flexible elongate element is conventionally called a hypotube. In one embodiment, the hypotube may have a length of 130 to 170 cm. Typically, such a guide wire may further include a stainless steel core wire extending from the proximal extremity to the distal extremity of the flexible elongate element to provide the desired torsional properties to facilitate steering of the guide wire in the vessel and to provide strength to the guidewire and prevent kinking.

In a preferred embodiment, methods of the invention employ a Doppler guidewire wire sold under the name FLOWIRE by Volcano Corporation, the pressure guidewire sold under the name PRIMEWIRE PRESTIGE by Volcano Corporation, or both.

The pressure and flow sensors allow one to determine whether the vascular access site is providing the proper amount of flow for hemodialysis. This includes both weak flow (constriction) and high flow rates. The constriction or lack of flow can be caused by stenosis, thrombosis, weakening vessel walls due to infection, and plaque. One of skill in the art will recognize that a high-flow fistula is associated with flow that is higher than what is best suited to maintain vascular access for hemodialysis. While any suitable criteria can be used for a high-flow fistula, in some embodiments, a flow rate >800 mL/min indicates a need for banding. This is considered in view of a target flow rate—i.e., a flow rate that is well-suited for hemodialysis. An exemplary target flow rate could be 600 mL/min. Existing medical guidelines provide that an adequate fistula has a flow >600 mL/min and a diameter >0.4 cm. A target flow rate is preferably less than about 800 mL/min (e.g., about 600 mL/min).

After the image data is processed to characterize biological material and/or foreign material (such as AV graft) in and around the vascular access site, the images and the characterization analysis are assessed in order to identify a condition and determine a therapeutic mode for treating the condition. In certain embodiments, functional flow measurements are assessed to assist in identifying the condition and determine course of treatment. For example, moderate to severe stenosis or thrombotic conditions identified at the vascular access site may require surgical invention (such as an atherectomy, angioplasty, or ablative therapy) alone or in combination with a therapeutic agent (e.g. thrombolytic, gene therapy (i.e., application of growth factors), or antineoplastic agent). In another example, vascular access sites (particularly fistulas) with heavy flow may require a banding procedure. In contrast, less severe stenosis thrombotic conditions identified at the vascular access site using the image and tissue characterization assessment may be treated with one or more therapeutic agents (e.g. thrombolytic agents, gene therapy (i.e., application of growth factors), antibiotics, or antineoplastic agents).

Suitable types of interventional procedures for treating a condition identified using the tissue characterization and image assessment of the invention are described hereinafter. One or more of the interventional procedures described herein may be used to treat the identified condition. Further, it is understood that course of treatment for various conditions identified using methods of the invention may vary and develop over time. Accordingly, methods of the invention encompass any therapeutic mode for treating a condition identified using methods of the invention.

In certain embodiments, the interventional procedure involves introducing a therapeutic agent to the vascular access site. A wide variety of therapeutic agents may be utilized within the scope of the present invention to inhibit formation of neointimal hyperplasia, including for example microtubule stabilizing agents, anti-proliferative agents including cytotoxic and cytostatic agents, anti-angiogenic agents, and the like (e.g., paclitaxel, or analogues or derivatives thereof), and other cell cycle inhibitors that may reduce the rate of cell proliferation. Furthermore, therapeutic drugs may include, but are not limited to, those agents that inhibit some or all of the processes involved in cell proliferation, cell migration, inflammation, and matrix deposition, such as in the development of intimal hyperplasia. In addition, therapeutic drugs may include, but are not limited to those agents that inhibit some or all of the processes involved in inflammation such as those involved in the development of intimal hyperplasia.

In one aspect, a therapeutic agent is introduced to the vascular access site that is capable of inhibiting smooth muscle cell migration, proliferation, matrix production, inflammation, or a combination thereof. Agents included in one or more of these categories are anti-angiogenic agents, e.g., anthracyclines (e.g., doxorubicin), fucoidon, and taxanes, and analogues or derivatives thereof; certain immunosuppressive compounds such as sirolimus (rapamycin), and analogues or derivatives thereof; certain anti-inflammatory agents, such as dexamethasone and analogues or derivatives thereof; certain antibiotic agents, e.g., dactinomycin and analogues or derivatives thereof; certain statins, such as cervistatin and analogues or derivatives thereof; and certain estrogens, e.g. 17-p-estradiol and analogues and derivatives thereof. Also included are those agents that have antithrombotic and/or antiplatelet properties such as clopidogrel, glycoprotein inhibitors (abciximab, eptifibatide, tirofiban and analogues and derivatives thereof. In other embodiments, a therapeutic agent is a thrombolytic drug or a growth factor. Each of these therapeutic agents may be used individually or in any combination thereof, and wherein some combinations results in synergistic effects. In addition, suitable therapeutic agents are described in U.S. Publication No. 2004/0146546. The therapeutic agents may be delivered or injected into the vascular access site using an interventional catheter or introduced into the vascular access site using a carrier device (such as a wrap, shunt, or band).

According to some embodiments, the interventional therapy includes introducing a perivascular wrap to the vascular access site. Typically the perivascular wrap is coated with a therapeutic agent and is designed to reduce formation of neointimal hyperplasia. Suitable perivascular wraps include a therapeutic agent and a mesh, wherein the mesh includes a biodegradable polymer. The mesh may be in the form of a woven, knit, or non-woven mesh. The therapeutic agents may be an integral part of the biodegradable polymer mesh (i.e., may reside within the fibers of the mesh) or may be coated on the mesh by painting, spraying, or dipping. The coated therapeutic agents may be in the form of a surface-adherent coating, mask, film, gel, foam, or mold. Perivascular wraps may be placed within the fistula or, in the case of AV grafts, at the graft-vein anastomosis

In certain embodiments, the interventional procedure is banding. Banding generally refers to procedures for restricting flow through a fistula. The introduction of a high-resistance band is a reasonable treatment for a low-resistance venous pathway, which has transformed a functional access into a pathologic shunt. Banding physiology is best explained by Poiseuille's law, which states that for laminar flow, volume flow rate Q is given by pressure drop across a gradient (ΔP) (e.g., arterial pressure-central venous pressure) divided by the viscous resistance R, where R is given by 8 μL/πr⁴, with μ being the fluid viscosity and r is the radius of the vessel. Thus, Q is ΔPπr⁴/8 μL. Banding techniques decrease flow by decreasing the radius at a specific point, and as a result, access flow and pressure are directly sacrificed to increase distal arterial flow and pressure. Any suitable banding technique can be used. Exemplary banding techniques include, for example, use of a narrowing suture, plication, minimally invasive limited ligation endoluminal-assisted revision (MILLER) banding, tapering, and surgical banding. See, e.g., West, et al., 1991, Arterial insufficiency in hemodialysis access procedures: correction by banding technique, Transpl Proc 23 (2): 1838-40; Rivers, et al., 1992, Correction of steal syndrome secondary to hemodialysis access fistulas: a simplified quantitative technique, Surgery 112(3):593-7; Kirkman, 1991, Technique for flow reduction in dialysis access fistulas, Surg Gyn Obstet 172(3):231-3; and Mickley, 2008, Steal Syndrome—strategies to preserve vascular access and extremity, Nephrol Dial Transplant 23:19-24.

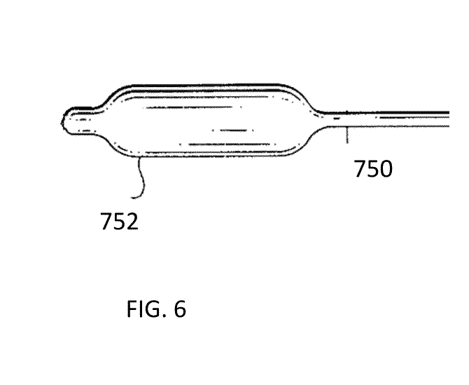

In further embodiments, the interventional therapy involves performing an angioplasty procedure. The angioplasty procedure may be performed by introducing an interventional balloon catheter. For angioplasty procedures, the inflatable balloon is introduced to a treatment site having plaque buildup and/or a thrombus. Inflation of the balloon disrupts and flattens the atheroma deposits and/or thrombus against the vessel wall, and stretches the vessel wall, resulting in enlargement of the vascular access passageway and increased blood flow. After such enlargement, the balloon is deflated, and the interventional catheter is removed. FIG. 6 shows the angioplasty tool suitable for use with methods of the invention that includes the elongate body 750 and inflatable balloon 752.

In yet another embodiment, the interventional therapy involves performing an atherectomy procedure. The atherectomy may be performed with an extraction tool. Atherectomy procedures involve removing atheroma, thrombus and other material blocking the vascular access site by mechanically breaking up and removing plaque/thrombus from the vessel lumen to re-canalizing blocked vascular access site. In certain embodiments, the extraction tool includes a distal end that can be extended from a lumen of an interventional catheter. The distal end of the extraction tool includes one or more cutting elements. Typically, a proximal portion of the extraction tool is formed as part of or operably coupled to a drive shaft. The drive shaft may be coupled to a motor to provide rotational motion using any conventional means. A drive shaft suitable for use to impart rotation of the extraction tool is described in, for example, U.S. Pat. No. 5,348,017, U.S. Patent Publication No. 2011/0306995, and co-assigned pending U.S. Publication No. 2009/0018393 (as applied to rotating imaging sensors). Rotation of the drive shaft causes rotation of the distal end of the extraction tool. In operation, the distal end of the extraction tool is deployed from the tool lumen of a catheter. Forward movement and/or rotation of the distal end of the extraction tool causes the one or more cutting element to engage with the plaque or other unwanted substances within a vessel. The cutting elements shave, morcellate, grind, or cut off plaque thrombosis, or other material blocking the vascular access site from the luminal surface to clear the occlusion within the fistula or AV graft.

In certain embodiments, the extraction tool of a catheter further defines a removal lumen extending from an opening located at the distal end of the extraction tool to an opening connected to a vacuum source. The vacuum source removes, via suction, plaque, thrombosis, or other material blocking the vascular access site that has been shaved, morcellated, or cut off from the luminal surface. Alternatively, a catheter itself may include a removal lumen that extends from the distal end of the imaging catheter to an opening operably associated with a vacuum source. In this embodiment, morcellated or shaved plaque/blood clot can be suctioned from the vessel through the removal lumen of the catheter.

The cutting elements used in the present invention will usually be formed from a metal, but could also be formed from hard plastics, ceramics, or composites of two or more materials, which can be honed or otherwise formed into the desired cutting edge. In certain embodiments, the cutting blades are formed as coaxial tubular blades with the cutting edges defined in aligned apertures therein. It will be appreciated that the present invention is not limited to any particular cutting element, and the cutting element may include a variety of other designs, such as the use of wiper blades, scissor blades or the like. The cutting elements can have razor-sharp smooth blade edges or serrated blade edges. Optionally, the cutting edge of either or both the blades may be hardened, e.g., by application of a coating. A preferred coating material is titanium nitride.

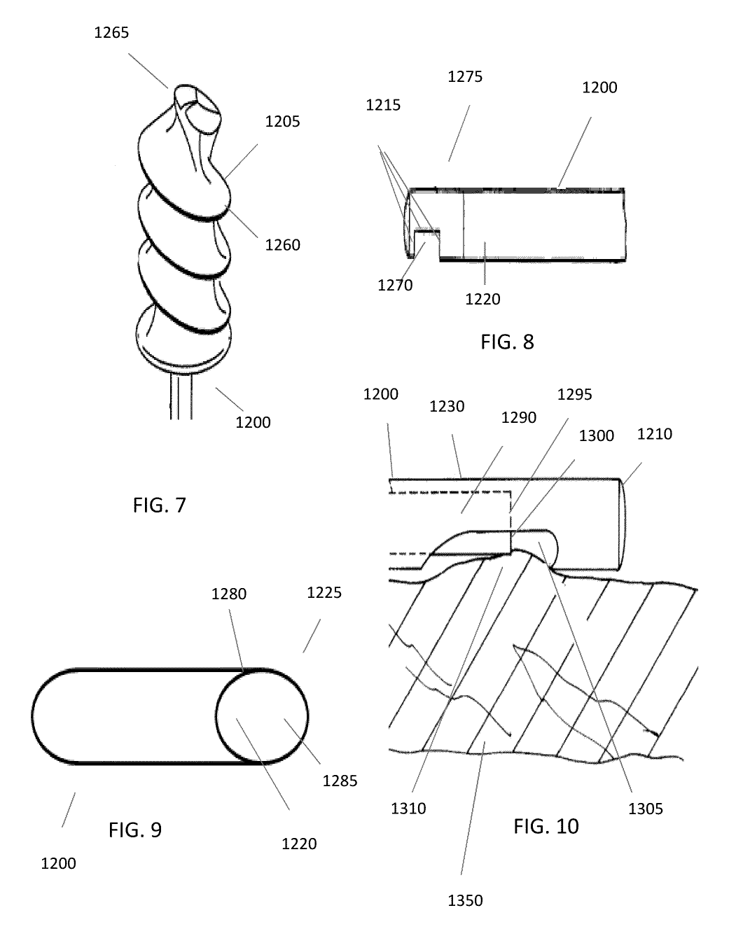

FIGS. 7-10 depict various embodiments of a distal end of the extraction tool suitable for use in methods of the invention. The extraction tool may be used alone or may be extended out of a catheter.

As shown in FIG. 7, the distal end 1200 of the extraction tool includes a helical cutting element 1205. The helical cutting element 1205 has a spiral-fluted shape. The edges 1260 of the spiral are sharp blades. When rotated, the helical cutting element 1205 grounds plaque within the vessel. The tip 1265 of the helical cutting element 1205 can be formed as a bladed point. The bladed point tip will assist in morcellating plaque/thrombosis that may be present in front of the extraction tool.

FIG. 8 depicts a distal end 1200 of an extraction tool according to one embodiment. The distal end 1200 of the extraction tool includes a recessed cutting element 1275. The recessed cutting element 1275 includes a recess 1260 within the distal end 1200 formed by edges 1260. One or more of the edges 1260 that form the recess 1260 constitute cutting blades. Optionally and as shown, the extraction tool includes a removal lumen 1220 and the recess 1260 provides access to the removal lumen 1220. The removal lumen 1220 can extend along the length of the extraction tool and operably couple to a vacuum source. In operation, the recessed cutting element 1275 is distally deployed from the tool lumen of the imaging catheter. The recessed cutting element 1275 can be moved forward and backwards and rotated to shave off or morcellate any plaque or unwanted substance that is placed within the recess 1260 via the blade edges 1260. The shaved off or morcellated material can be removed from the vessel through the removal lumen 1220.

FIG. 9 depicts a distal end 1200 an extraction tool according to another embodiment. The extraction tool includes a tubular member with a bladed end 1225 at the distal end 1220. The bladed end 1225 is formed by a sharp edge 1280. The bladed end 1225 can be open or closed. As shown in FIG. 9, the bladed end is open and includes opening 1285. The opening 1285 leads to a removal lumen 1220. In order to morcellate plaque and other unwanted substances, the distal end 1200 of extraction tool is deployed from the tool lumen of the imaging catheter. As the distal end 1200 is moved forward and rotated, the sharp edge 1280 cuts through and morcellates unwanted material (plaque/thrombus) present in front of the distal end 1200. The shaved off or morcellated material can be removed from the vessel through the removal lumen 1220.

FIG. 10 depicts the distal end 1200 of an extraction tool according to yet another embodiment. The extraction tool includes an outer tubular member 1210 that defines a removal lumen 1230 and an inner tubular member 1290 disposed within the removal lumen 1230. The outer tubular member 1210 includes a window 1305. The removal lumen 1230 can be operably coupled to a vacuum source. The inner tubular member 1290 can be moved forward and backward and rotated with respect to the outer tubular member 1210. The inner tubular member includes the same elements as the extraction tool shown in FIG. 9. The inner tubular member 1290 includes a bladed end 1295. The bladed end 1295 can be open or closed. The bladed end 1295 is formed by a sharp edge 1300. In operation, the distal end 1200 of the extraction tool is deployed from the tool lumen of the imaging catheter. The window 1305 of the outer tubular member 1210 is placed against plaque 1310 protruding from the vessel wall 1350. The inner tubular member 1290 can be moved forward and backwards and rotated within outer tubular member to morcellate and shave off any plaque placed within the window 1305. Removed plaque can be suctioned out of the vessel through the removal lumen 1230.

In further embodiments, the interventional treatment includes apply ablative energy to the vascular access site. The ablative therapy is designed to inhibit further formation of hyperplasia in vascular fistulas and grafts. The ablative energy can be provided from a number of sources including radiofrequency, laser, microwave, ultrasound and forms of direct current (high energy, low energy and fulgutronization procedures). Radiofrequency (RF) has become the preferred source of energy for ablation procedures. Any source of energy is suitable for use in the ablation tool of the invention. Catheters for administering ablative energy are known in the art and are describe in, e.g., U.S. Pat. Nos. 8,486,063 and 8,486,062 as well as U.S. Publication Nos. 20130137980 and 20130296704.

According to certain embodiments, methods of the invention further include assessing the vascular site after the interventional procedure (see step 109). The intraluminal image data can be obtained with any one of the imaging catheters (e.g., IVUS or OCT) described above, or the intraluminal image data can be obtained from, for example, an imaging element located on the interventional catheter. The intraluminal image data is then reviewed to determine the success of the interventional therapy. In certain embodiments, the intraluminal image data of the treated vascular access site is processed to characterize biological and/or foreign material present at the vascular access site. The images and characterization can then be assessed in order to identify whether a condition still exists at the treated vascular access site, and, if a condition exists, to determine if further treatment is necessary to treat the identified condition. Any of the above therapeutic modes for treating a vascular access site can be used for the further treatment. This process can be repeated until the identified condition is fully treated.

In further embodiments, methods of the invention also provide for long-term follow up assessments to continually monitor the treated vascular access site. For example, the follow-up assessments may be scheduled for 3, 6, 9, and 12 months after the intervention therapy. The long-term follow up assessments repeat the method outlined in FIG. 1 in order to identify a condition at the vascular image site and determine course of treatment.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for assessing a vascular access site for hemodialysis, the method comprising
processing intraluminal image data of a vascular access site to characterize biological material present at the vascular access site;
assessing the characterized biological material to identify a condition; and
determining a therapeutic mode for treating the condition based on the assessment.

2. The method of claim 1, further comprising delivering an interventional catheter to the vascular access site; and treating the condition with the interventional catheter.

3. The method of claim 1, further comprising processing the intraluminal image data to characterize non-biological material at the vascular access site.

4. The method of claim 3, wherein the non-biological material is a graft.

5. The method of claim 1, wherein the processing comprises determining imaging assembly-position-dependent changes in a spectrum of the image data.

6. The method of claim 1, wherein the processing comprises determining a density of the biological material.

7. The method of claim 1, wherein the processing comprises determining a composition of the biological material.

8. The method of claim 1, wherein the processing comprises determining a blood-tissue border of a lumen of the vascular access site.

9. The method of claim 8, further comprising displaying an image comprising a cross section of the lumen of the vascular access site and the determined blood-tissue border of the lumen of the vascular access site.

10. The method of claim 9, further comprising colorizing the image to show the blood-tissue border of the lumen.

11. The method of claim 10, wherein the colorizing comprises displaying an area within the blood-tissue border of the lumen as red.

12. The method of claim 1, wherein the biological material is thrombotic, sclerotic, or a combination thereof.

13. The method of claim 1, wherein the condition is stenosis, thrombosis, or a combination thereof.

14. The method of claim 1, wherein the therapeutic mode for treating the condition is selected from the group of performing an angioplasty procedure, introducing a therapeutic agent to the vascular access site, performing an atherectomy procedure, banding the vascular access site, introducing a perivascular wrap, and a combination thereof.

15. The method of claim 1, wherein the vascular access site is a fistula or a graft.

16. A method for assessing and treating a vascular access site for hemodialysis, the method comprising
introducing an imaging catheter into a lumen of a blood vessel;
imaging, with the imaging catheter, a vascular access site within the blood vessel to generate image data;
processing the image data to characterize biological material at the vascular access site;
assessing the characterized biological material in order to identify a condition and determine a therapeutic mode for treating the condition;
introducing an interventional catheter to the vascular access site based on the assessing step; and
treating the condition with the interventional catheter.

17. The method of claim 16, wherein the imaging catheter comprises an elongate body and an imaging assembly operably associated with the elongate body, wherein the imaging assembly is selected from the group consisting of an ultrasound assembly and optical imaging assembly.

18. The method of claim 16, further comprising processing the image data to characterize non-biological material at the vascular access site.

19. The method of claim 18, wherein the non-biological material is a graft.

20. The method of claim 16, wherein the processing comprises determining imaging assembly-position-dependent changes in a spectrum of the image data.

21. The method of claim 16, wherein the processing comprises determining a density of the biological material.

22. The method of claim 16, wherein the processing comprises determining a composition of the biological material.

23. The method of claim 16, wherein the processing comprises determining a blood-tissue border of the lumen of the vessel.

24. The method of claim 23, further comprising displaying an image comprising a cross section of the lumen of the vessel and the determined blood-tissue border of the lumen of the vessel.

25. The method of claim 24, further comprising colorizing the image to show the blood-tissue border of the lumen.

26. The method of claim 25, wherein the colorizing comprises displaying an area within the blood-tissue border of the lumen as red.

27. The method of claim 16, wherein the biological material is thrombotic, sclerotic, or combination thereof.

28. The method of claim 16, wherein the imaging catheter further comprises a data collector configured to receiver functional flow data within the blood vessel.

29. The method of claim 28, wherein the data collector is selected from the group consisting of a flow sensor, a pressure sensor, and a combination thereof.

30. The method of claim 16, wherein the condition is stenosis, thrombosis, or a combination thereof.

31. The method of claim 16, wherein the therapeutic mode for treating the condition is selected from the group of performing an angioplasty procedure, introducing a therapeutic agent to the vascular access site, performing an atherectomy procedure, introducing a vascular wrap, banding the vascular access site, and a combination thereof.

32. The method of claim 16, wherein the vascular access site is a fistula or a graft.
